(19) United States
(12) Patent Application Publication
(10) Pub. No.: US 2006/0035214 A1
(43) Pub. Date: Feb. 16, 2006

(54) Screening methods for identifying inhibitors of herpesviral replication

(75) Inventors: Sheng-Hao Chao, Vienna, VA (US); Jeremy Caldwell, La Jolla, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, a Delaware Limited Liability Company, Hamilton (BM)

(21) Appl. No.: 11/115,838

(22) Filed: Apr. 26, 2005

Related U.S. Application Data
(60) Provisional application No. 60/566,454, filed on Apr. 29, 2004.

(51) Int. Cl. C12Q 1/70

(52) U.S. Cl. 435/5

(57) ABSTRACT

This invention provides methods of screening for compounds that inhibit herpesviral transcription and replication. The methods comprise screening test compounds for ability to enhance the activity of homeodomain transcription factor SATB1 or CDP in repressing transcription of herpesviral genes (e.g., the IE gene of cytomegalovirus). Transcriptional repression by SATB1 or CDP can be monitored using an expression vector comprising a reporter gene operably linked to an SATB1/CDP-binding transcription regulatory sequence of the herpesvirus. The invention further provides methods and pharmaceutical compositions for stimulating SATB1 or CDP-mediated transcriptional repression in a subject and for treating diseases and conditions associated with herpesviral infection.

### CROSS-REFERENCE TO RELATED APPLICATIONS

This application claims the benefit of priority under 35 U.S.C. §119(e) to U.S. Provisional Patent Application No. 60/566,454, filed Apr. 29, 2004. The disclosure of the priority application is incorporated herein by reference in its entirety and for all purposes.

### FIELD OF THE INVENTION

This invention relates to the discovery that the transcription factors SATB1 and CDP play an important role in repressing transcription of the immediate early (IE) gene of cytomegalovirus (CMV). The invention accordingly provides methods for identifying novel compounds that enhance the transcription repressing activity of SATB1 and/or CDP. Such novel modulators can be used to modulate SATB1 and CDP-mediated transcriptional repression of herpesviral genes and to inhibit replication of herpesviruses, e.g., CMV.

### BACKGROUND OF THE INVENTION

Members of the herpesviridae family are enveloped, double-stranded DNA viruses with relatively large complex genomes. At least 8 human herpesviruses have been identified, including human herpesvirus 1, 2 (HSV-1, HSV-2), varicellovirus (VZV) or human herpesvirus 3, cytomegalovirus (CMV) or human herpesvirus 5, roseolovirus or human herpesvirus 6, 7 (HHV-6, HHV-7), lymphocryptovirus (Epstein-Barr virus or EBV) or human herpesvirus 4, and rhadinovirus or human herpesvirus 8. These viruses are implicated in a number of human diseases such as facial or genital lesions and lymphotropic conditions. Human herpesvirus infections are endemic, and sexual contact is a significant method of transmission for several herpesviruses including herpes simplex virus 1 and 2, human CMV and likely Karposi's sarcoma herpesvirus. Prevalence of genetial herpes and corresponding rise of neonatal infection have implicated Epstein-Barr virus and Karposi's sarcoma herpesvirus as cofactors in human cancers.

Among herpesviruses, human CMV is a significant opportunistic pathogen responsible for serious clinical consequences in a variety of human subjects. Human subjects affected by CMV include immunosuppressed patient groups such as neonate and infants, persons with AIDS and individuals undergoing immunosuppressive regimes for the purpose of organ or bone marrow transplantation. Like other human herpesviruses, CMV establishes a life-long latent infection with its human host and is ubiquitous in the population with upwards of 75% infectivity rate found in the United States.

The currently available drugs for treating infections of herpesviruses are not very satisfactory due to various reasons. There is a need in the art to identify alternative drug targets and better agents to treat diseases and conditions associated with herpesviruses, e.g., CMV infection. The instant invention fulfills this and other needs.

### SUMMARY OF THE INVENTION

In one aspect, the present invention provides methods for identifying modulators that inhibit transcription and replication of herpesviruses. The methods comprise screening test compounds for ability to enhance SATB1 or CDP-mediated transcriptional repression of the herpesvirus, thereby identifying a compound that inhibits replication of the herpesvirus. Some of the methods are directed to identifying modulators that inhibit transcription and replication of human cytomegalovirus (CMV).

In some methods, the screening entails (a) contacting test compounds with an SATB1 or a CDP polypeptide, and a polynucleotide comprising an SATB1/CDP response element; and (b) identifying a compound that enhances binding to the SATB1/CDP response element by the SATB1 or the CDP polypeptide relative to the binding in the absence of the compound. In these methods, the compound thus identified can be further tested for ability to inhibit replication of the herpesvirus.

In some methods, the screening entails (a) contacting test compounds with an SATB1 or a CDP polypeptide, and a gene under the control of an SATB1/CDP response element, and (b) identifying a compound that reduces expression level of the gene relative to expression level of the gene in the absence of the compound. In some of these methods, the test compounds are pre-screened for ability to specifically bind to the SATB1 or the CDP polypeptide. In some other methods, expression level of the gene is measured using a reporter construct comprising the SATB1/CDP response element operably linked to a polynucleotide that encodes a detectable label. In some of the methods, the SATB1/CDP response element comprises a transcription regulatory sequence from the IE gene of the herpesvirus. The transcription regulatory sequence that can be used can be nucleotides −593 to −549 (SEQ ID NO: 1), nucleotides −735 to −688 (SEQ ID NO: 3), or nucleotides −687 to −640 (SEQ ID NO: 4) of human CMV IE gene promoter. In some methods, the herpesvirus is human cytomegalovirus (CMV), and the SATB1 or CDP polypeptide is human SATB1 or CDP.

In a related aspect, the invention provides novel methods for identifying a compound that inhibits replication of a herpesvirus that infects human cells. These methods involve (a) contacting test compounds with an SATB1 or a CDP polypeptide, and a reporter gene operably linked to an SATB1/CDP response element, and (b) identifying a compound that reduces expression level of the reporter gene in the presence of the compound relative to expression level of the reporter gene in the absence of the compound. The SATB1/CDP response element can contain a transcription regulatory sequence from the IE gene of the herpesvirus. For example, it can be nucleotides −593 to −549 (SEQ ID NO: 1), nucleotides −735 to −688 (SEQ ID NO: 3), or nucleotides −687 to −640 (SEQ ID NO: 4) of human CMV IE gene promoter.

In some of these methods, the reporter gene and the SATB1/CDP response element are present in an expression vector. In some methods, the contacting takes place in a host cell expressing the SATB1 and the CDP polypeptide. The SATB1 and the CDP polypeptide can be expressed from a second expression construct that has been introduced into the host cell. They can also be expressed by the host cells endogenously. In some methods, the reporter gene used is a luciferase gene. An example of the host cell that can be used is HEK 293 cell.

In another aspect, methods for inhibiting replication of a herpesvirus in a subject are provided. These methods entail administering to the subject a pharmaceutical composition comprising an effective amount of a compound that inhibits replication of the herpesvirus. The compound used in these methods is identified by screening test compounds for ability to enhance SATB1 or CDP-mediated transcriptional repression of the herpesvirus. Some of these methods are directed to inhibiting replication of human CMV.

A further understanding of the nature and advantages of the present invention may be realized by reference to the remaining portions of the specification and claims.

### BRIEF DESCRIPTION OF THE DRAWINGS

FIG. 1 shows diagram of the human CMV IE promoter. The locations of the transcription start sites (+1), enhancer (−118 to −524), and the unique region (−735 to −550) are indicated. The 45-bp PDX1 binding region (−593 to −549) contains a potential human HPV silencing motif (boxed) and a putative Brn-3.0 binding site (underlined).

FIG. 2 shows that SATB1 and CDP associate with the 45-bp PDX1 binding region in 293 cells. EMSAs were performed using nuclear extracts of 293 cells and the radiolabeled CMV_{-593 to -549} DNA probe. “C1” and “C2” denote the specific DNA-protein complexes formed in the presence of the CMV DNA probe and 293 nuclear extracts. While the unlabeled specific competitor (i.e. unlabeled CMV_{-593 to -549} oligonucleotide; S.C.) was included, both DNA-protein complexes were disrupted. No effects on C1 or C2 were observed with unlabeled nonspecific competitor (i.e. unlabeled SP1 consensus oligonucleotide; N.S.C.). Nuclear extracts of 293 cells were incubated with antibodies against PDX1, CDP, SATB1, Brn-3.0 and SP1 prior to addition of the CMV probe. The anti-SP1 antibody was used as the negative control.

FIG. 3 shows that in vitro synthesized SATB1 and CDP proteins associate with the 45-bp PDX1 binding region. EMSA was carried out using the CMV_{-593 to -549} probe (which contains the entire 45-bp CMV element from −593 to −549) and in vitro translated PDX1, SATB1, and CDP proteins produced by a coupled reticulocyte lysate system. The first lane contains reticulocyte lysates alone and does not include an expression construct, thus demonstrating the binding of endogenous complexes in lysates. The arrows indicate the binding of PDX1, SATB1, and CDP proteins.

FIG. 4 shows that in vitro synthesized PDX1, SATB1, and CDP proteins bind to multiple sites located within the unique region of the human CMV IE promoter. The unique region of the CMV IE promoter was equally divided into four segments extending from position −735 to −688 (CMV_{-735 to -088}), −687 to −640 (CMV_{-687 to -640}), −639 to −594 (CMV_{-639 to -594}), and −593 to −549 (CMV_{-593 to -549}) EMSAs were performed by incubating the in vitro synthesized PDX1, SATB1, and CDP proteins, and the indicated CMV DNA probe. The lanes containing reticulocyte lysates alone, which do not include any expression plasmid, served as negative controls. The arrows indicate the binding of PDX1, SATB1, and CDP proteins.

FIG. 5 shows that PDX1, SATB1, and CDP proteins bind to the first half of the unique region in 293 cells. EMSAs were performed using nuclear extracts of 293 cells and the indicated DNA probe (CMV_{-735 to -688}, CMV_{687 to -640}, or CMV_{-639 to -594}). The specific antibodies were included in the reactions before adding the DNA probe (lanes 6-9, 14-17, and 23-26). Lanes 1, 10, and 18 contained the DNA probe alone. The probe and nuclear extracts were included in lanes 2, 5, 11, 19, and 22. Lanes 3, 12, and 20 contained the specific competitor (i.e. unlabeled CMV_{-735 to -688}, CMV_{-687 to -640}, or CMV_{-639 to -594} oligonucleotide, respectively; S.C.), whereas lanes 4, 13, and 21 contained the nonspecific competitor probes (i.e. unlabeled SP1 consensus oligonucleotide; N.S.C.). Two protein-CMV_{-735 to -688} (i.e. C1* and C2*), one protein-CMV_{-687 to -640} (i.e. C3), and two protein-CMV_{-639 to -594} (i.e. C4 and C5) complexes were indicated.

FIGS. 6A-6B show that PDX1, CDP, and SATB1 additively repress the CMV IE-dependent transcription. (A) Inhibitory effects of PDX1, CDP, and SATB1 overexpression on the CMV IE promoter. 293T cells were transiently transfected with CMV-R-Luc (CMV-Renilla luciferase), pUB-F-Luc (ubiquitin-firefly luciferase), and 0.1 μg (black columns) or 0.3 μg (white columns) of pUB-PDX1, pUB-CDP, or pUB-SATB1 plasmids individually. pUB-F-Luc was used to normalize transfection efficiency. The activity of Renilla and firefly luciferases was determined by Dual-Glo assays 2 days after transfection. (B) Additive inhibition of PDX1, CDP, and SATB1 on the CMV IE promoter. 293T cells were transiently co-transfected with CMV-R-Luc, pUB-F-Luc, and the indicated combinations of 0.1 μg PDX1, CDP, and SATB1 expression vectors.

FIG. 7 shows that deletion of the unique region results in the alleviation of the PDX1/SATB1/CDP-mediated repression on the human CMV IE promoter. 293 cells were transiently transfected with the indicated combinations of 0.1 μg PDX1, CDP, and SATB1 vectors, as well as the CMV₋₇₃₅-Luc (black columns) or CMV₋₅₄₉-Luc (white columns) reporter plasmid. The luciferase activity was measured 2 days after transfection.

FIG. 8 shows that differentiation status of monocytes affects the formation of the specific DNA-protein complexes at the CMV_{-593 to -549} region. THP-1, a human monocytic cell line, was induced to differentiate into macrophage-like cells through treatment with 10 nM PMA for 2 days. EMSAs were performed using the nuclear extracts prepared from undifferentiated (THP-1) and differentiated (THP-1+PMA) monocytes, and the radiolabeled CMV_{-593 to -549} as the DNA probe. Two specific DNA-protein complexes formed in undifferentiated THP-1 cells were labeled as S1 and S2. The newly formed DNA-protein complex due to the induced differentiation was indicated by an arrow.

FIGS. 9A-9B show that PDX1, SATB1, and CDP proteins bind to the first half of the unique region in undifferentiated and differentiated THP-1 cells. (A) EMSAs were carried out using the nuclear extracts of untreated (THP-1) or PMA-treated (THP-1+PMA) THP-1 cells. CMV_{-735 to -688} was used as the DNA probe. Two specific DNA-protein complexes formed in undifferentiated THP-1 cells were labeled as S1* and S2*. The differentiation-induced DNA-protein complex was indicated by an arrow. (B) The same set of EMSAs were performed using CMV_{-687 to -640}, instead of CMV_{-735 to -688}, as the DNA probe. Two specific DNA-protein complexes, which contain PDX1, SATB1, and CDP, were denoted as M1 and M2.

### DETAILED DESCRIPTION OF THE INVENTION

I. Introduction

The invention is based in part on the discovery that two cellular homeoproteins, SATB1 or CDP, function as transcriptional repressors of the immediate early (IE) gene of human cytomegalovirus (CMV). STAB1, or special AT-rich sequence binding protein 1, is a DNA-binding protein expressed predominantly in thymocytes. It is a cell-type specific nuclear protein that recruits chromatin-remodeling factors and regulates numerous genes during thymocyte differentiation. SATB1 recognizes and binds in a specific AT-rich sequence context where one strand consists of mixed A's, T's, and C's excluding G's (ATC sequences). CDP, also termed “CCAAT displacement protein,” CDP-Cut, CUTL1, or Cux, is the mammalian homolog of the Drosophila homeoprotein CUT. It has been implicated in the pathogenesis of diverse virus families. CDP has been shown to negatively regulate human papillomavirus (HPV) dependent transcription by binding to specific DNA elements located in the viral promoters and long control regions, thus repressing HPV replication. The transcription inhibitory effects of CDP have also been demonstrated on mouse mammary tumor viruses (MMTV) transcription. By virtue of an interaction with SATB1, CDP has been shown to bind to the negative regulatory elements located in the long terminal repeats of MMTV.

The IE gene expression is the key to latency and active replication of herpesviruses due to its transactivating and repressing functions. It was found that pancreatic-duodenal homeobox factor-1 (PDX1) associates with a specific 45-bp element located in the unique region of the CMV IE promoter, and that PDX1 represses the CMV IE-dependent transcription in the cell-based assays (J Biol. Chem. 2004 Feb. 5 [Epub ahead of print]). PDX1 has also been shown to function as a transcriptional activator of several genes, including insulin, Glut2, glucokinase, islet amyloid polypeptide, and somatostatin.

The present inventors identified two transcriptional repressors, SATB1 and CDP homeoproteins, that associate with the 45-bp PDX1 binding element of the CMV IE promoter. In addition, more PDX1/SATB1/CDP binding sites located in the different parts of the CMV IE unique region were identified by gel shift assays. It was further demonstrated the additive repression of PDX1/SATB1/CDP on the activity of the CMV IE promoter in cell-based assays. The CMV-Luc construct with the deletion of the entire unique region was generated and the effects of the deletion on CMV IE dependent expression were examined in the cell-based assays. As expected, deletion of the CMV IE unique region alleviated the inhibitory effects of PDX1/SATB1/CDP proteins on the CMV IE promoter. Interactions between the unique region and PDX1/SATB1/CDP were examined in the CMV host cells. Interestingly, different DNA-PDX1/SATB1/CDP protein complexes were observed in the undifferentiated and differentiated monocytes, suggesting possible involvement of the three proteins in transcriptional regulation of the CMV IE gene. These studies demonstrated that three cellular homeoproteins, PDX1, SATB1, and CDP, associate with the multiple sites located in the unique region of the human CMV IE promoter and regulate the promoter activity.

In accordance with these discoveries, the present invention provides methods for identifying agents that enhance SATB1 or CDP-mediated transcription repressing activity and thus inhibit replication of herpesviruses (e.g., CMV) that infect human cells. This invention relies on routine techniques in the field of recombinant genetics. Basic texts disclosing the general methods to be employed in the invention include Sambrook et al., Molecular Cloning, A Laboratory Manual (3 rd ed. 2001); Kriegler, Gene Transfer and Expression: A Laboratory Manual (1990); and Current Protocols in Molecular Biology (Ausubel et al., eds., 1994). The following sections provide further guidance for making and using the compositions of the invention, and for carrying out the methods of the invention.

II. Definition

Unless defined otherwise, all technical and scientific terms used herein have the same meaning as commonly understood by those of ordinary skill in the art to which this invention pertains. The following references provide one of skill with a general definition of many of the terms used in this invention: Singleton et al., Dictionary of Microbiology and Molecular Biology (3d ed. 2002); the Larousse Dictionary of Science and Technology (Walker ed., 1995); and the Collins Dictionary of Biology (2d ed. 1999). In addition, the following definitions are provided to assist the reader in the practice of the invention.

A “full length” SATB1 or CDP protein or SATB1 or CDP polynucleotide refers to a polypeptide or polynucleotide, or a variant thereof, that contains all of the elements normally contained in one or more naturally occurring, wild type SATB1 or CDP polynucleotides or SATB1 or CDP polypeptides. It will be recognized, however, that derivatives, homologs, and fragments of an SATB1 or CDP that modulate a herpesvirus (e.g., human CMV) replication can be readily used in the present invention.

The term “inhibiting replication of a herpesvirus that infects human cells” as used herein refers to inhibition of any process in human herpesvirus replication. The term includes, but is not limited to, processes such as transcription of herpesviral genes. Other processes that may be involved in the replication of herpesvirus include viral integration, RNA processing, and assembly of virus particles. Typically, the methods of the invention identify modulators that inhibit herpesviral gene transcription.

“Modulators” refer to molecules that enhance the ability of SATB1 or CDP to suppress replication of a herpesvirus (e.g., CMV) that infects human cells. Typically, such modulators promote or enhance transcriptional repression mediated by SATB1 or CDP. Modulators include naturally occurring and synthetic compounds. Samples or assays comprising an SATB1 or CDP polypeptide or SATB1 or CDP nucleic acid sequence and a test compound as described herein are treated with a potential modulator and are compared to control samples without the modulator to examine the extent of effect. Control samples (not treated with modulators) are assigned a relative activity value of 100%. Modulation of SATB1 or CDP activity is achieved when the activity (e.g., transcriptional repression activity) compared to the control is more than about 20%, optionally 50%, or 100%, 200%, 500% or more.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

The term “identical” in the context of two or more SATB1 or CDP polynucleotide or polypeptide sequences, refer to two or more sequences or subsequences that are the same sequences. Two sequences are substantially identical if the two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. The invention can employ SATB1 or CDP polypeptides or polynucleotides that are substantially identical to the human SATB1 or CDP polypeptide or SATB1 or CDP polynucleotide, respectively, exemplified herein. Optionally, the identity exists over a region that is at least about 50 nucleotides or amino acids in length, or more preferably over a region that is 100 to 500 or 1000 or more nucleotides or amino acids in length.

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” or “expression construct” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

III. SATB1 and CDP-CMV IE Transcriptional Repressors

Expression of the human CMV IE genes plays a critical role in production of infectious CMV. Most studies have been focused on characterization of the CMV IE enhancer region (between nucleotides −524 and −118) and identification of the transcription activators associated with the element. However, little is known about biological function of the unique region (from position −735 to −549) and identities of the cellular proteins which bind to the region. A cellular homeoprotein, PDX1, binds to a specific 45-bp element located within the unique region (which spans nucleotides from −593 to −549) of the CMV IE promoter, and down-regulates the promoter activity in 293/293T cells (Chao et al., J Biol. Chem., 279: 16111-120, 2004).

The present inventors found that two additional cellular homeoproteins, SATB1 and CDP, are also involved in transcriptional repression of CMV IE gene. CDP is a known transcriptional repressor and has been shown to negatively regulate numerous cellular genes and viral transcription of HPV and MMTV. SATB1, which interacts with CDP in vivo, is also involved in repression of MMTV transcription. Interaction between SATB1 and CDP proteins in vivo has been described in Liu et al., Mol. Cell Biol. 19: 4918-4926, 1999. It was found that these two proteins associate with the 45-bp PDX1-binding region. Additional binding sites were also found for PDX1, SATB1, and CDP upstream of the 45-bp element (between nucleotides −735 and −640). Overexpression of the three homeodomain proteins resulted in additive inhibition of the CMV IE promoter in 293/293T cells, suggesting functional interaction between the proteins. EMSA studies showed interactions between the unique region and PDX1/SATB1/CDP in human CMV monocyte host cells. Cells of the monocyte lineage serve as reservoirs of latent human CMV and as vehicles for disseminating viral infection. It has been shown that the expression of IE genes is turned off in undifferentiated monocytes, but reactivated once the monocytes are differentiated to macrophages.

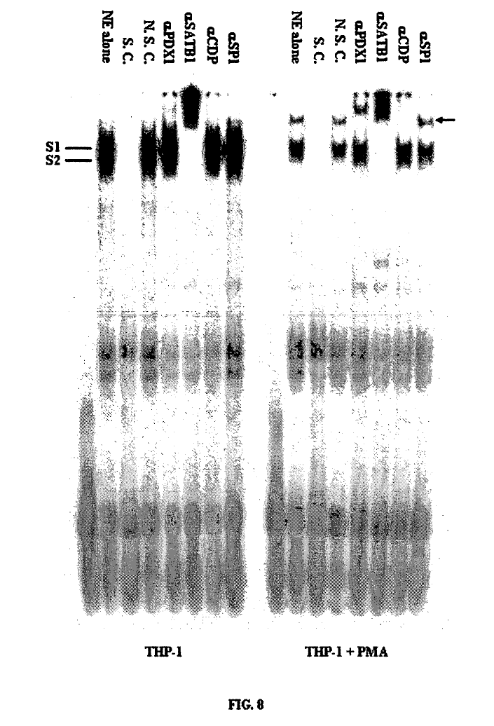

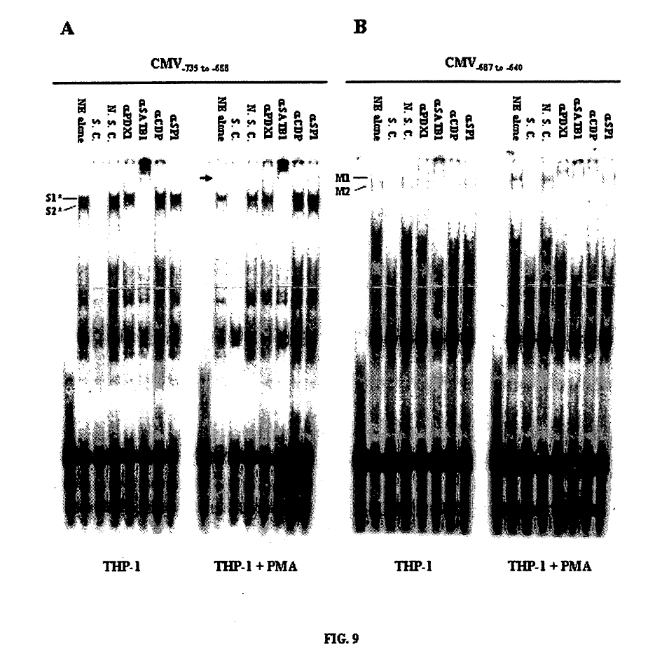

As detailed in the Examples below, the present inventors observed a correlation between regulation of CMV IE genes and expression profiles of SATB1 and CDP. The present inventors also observed that differentiation status of human monocytes results in the formation of novel DNA-protein complexes. Studies of the present inventors showed that SATB1 could be the major cellular factor which regulates the transcriptional repression of human CMV IE genes in human host cells (e.g., undifferentiated monocytes). First, as shown in FIGS. 8 and 9, SATB1 is the major cell factor binding to the unique region in undifferentiated monocytes. Since the CMV IE genes remain transcriptionally silent in undifferentiated monocytes, these data indicate that SATB1 may play an important role in transcriptional repression of human CMV IE genes. In addition, the present inventors examined activity of the CMV IE promoter in Jurkat cells transiently transfected with CMV-Luc or pUB-Luc. The luciferase activity of the pUB-Luc transfected cells was more than 50-fold than that of the CMV-Luc transfected cells, suggesting the possible involvement of SATB1 in CMV IE repression. Further, expression of SATB1 in THP-1 and Jurkat cells was also examined by western blot. The results showed that the levels of SATB1 proteins in monocytes were higher than those in T cells.

IV. Screening for Novel SATB1/CDP Modulators—General Scheme

The inhibitory activities of SATB1 and CDP on CMV IE-mediated transcription allow one to identify novel modulators of CMV transcription and replication. Such novel modulators (e.g., inhibitors) can be identified by screening test compounds for agents that can alter SATB1 and/or CDP activities on CMV IE-mediated transcription. Test compounds or candidate agents that can be employed to screen for modulators of SATB1 or CDP activity include any chemical compound or other biological entities, e.g., proteins, lipids, saccharides, fatty acids, steroids, purines, pyrimidines, nucleic acids, and various structural analogs or combinations thereof. They also include antibodies or fragments, and extracts from natural sources. In some preferred embodiments, test compounds to be screened in the present invention are small organic molecules which generally having a molecular weight of more than about 100 and less than about 2,500 daltons. Typical small molecules are less than about 2,000, less than about 1,500, less than about 1,000, or less than about 500 daltons.

The test compounds typically include functional groups necessary for structural interactions with proteins or nucleic acids, e.g., hydrogen bonding, and typically include at least an amine, carbonyl, hydroxyl, or carboxyl group, preferably at least two of the functional chemical groups. The candidate agents often comprise cyclical carbon or heterocyclic structures and/or aromatic or polyaromatic structures substituted with one or more of the above functional groups. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

The screening methods of the invention can be designed to screen large chemical libraries by automating the assay steps, which are typically run in parallel (e.g., in microtiter formats on microtiter plates in robotic assays). In preferred embodiments, high throughput screening methods are employed. These methods involve providing a combinatorial library, e.g., a chemical library, containing a large number of potential therapeutic compounds. Such “combinatorial chemical libraries” are then screened in one or more assays, such as transcriptional assays as described herein, to identify those library members (particular chemical species or subclasses) that display the desired characteristic activity, e.g., stimulation of SATB1 or CDP-mediated transcriptional repression. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel & Sambrook, both supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Employing high throughput assays of the invention, it is possible to screen thousands of different modulators in a single day. In particular, each well of a microtiter plate, e.g., a 96, 384, or 1,536-well plate, can be used to run a separate assay against a selected potential modulator, or, if concentration or incubation time effects are to be observed, every 5-10 wells can test a single modulator. Thus, a single standard microtiter plate can assay a large number of modulators. For example, if 1536-well plates are used, a single plate can easily assay from about 100-1500 different compounds. It is possible to assay many different plates, for example over 1 million wells per days, using high throughput systems, e.g., those described in WO02/31747. Thus, many thousands of compounds can be screened in a single day.

High throughput systems comprise automated components, including fluid transfer and dispensing devices. A number of fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to microtiter plates to set up several parallel simultaneous assays. Such a fluid transfer device typically comprises an array of receptacles arranged such that the outlets of the receptacle are aligned with wells on the microwell plate. The Robbins Hydra (Robbins, Scientific, Sunnyvale, Calif.) is another example of a fluid dispensing device that can also be used in high throughput screening systems. Other fluid manipulation devices may include those that incorporate positive displacement pumps and dispenser valves, such a Cartesian SynQUAD (U.S. Pat. No. 6,063,339, available from Cartesian Technologies, Inc., Irvine, Calif.).

As appreciated by one of skill in the art, the high throughput devices used in the screening methods may also comprise additional components such as an incubator, e.g., to provides particular growth conditions for cells. Detectors may also be included in the high throughput assay system. The detectors may measure any physical property of a sample. For example, fluorescence, luminescence, phosphorescence, radioactivity, or any other physical property may be measured by the detector. Examples of detectors that are often used in cell-based high throughput screening assays include a Fluormetric Imaging Plate Reader System (FLIPR®), which is commercially available from Molecular Devices Corp. Sunnyvale, Calif.; and a chemiluminescent imaging plate reader (CLIPR™). Additional imaging systems are described, e.g., in WO00/17643.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image for high throughput systems.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. This type of apparatus is easily used for viewing any sample, e.g., by fluorescent or dark field microscopic techniques.

V. Screening for Compounds that Bind to SATB1 or CDP

To identify novel modulator of herpesviral transcription and replication, test compounds can be screened directly for ability to enhance SATB1 or CDP-mediated transcriptional repression of herpesviral genes. Alternatively, test compounds can be examined for their ability to bind to SATB1 or CDP, and the binding assays are typically performed in conjunction with transcription assays. The binding and transcription assays may be performed in either order. In some embodiments, test compounds are first screened with a binding assay. Such pre-screening allows identification of compounds that specifically bind to an SATB1 or a CDP polypeptide. These compounds can then be used to identify modulators that enhance transcription repressing activity of SATB1 and/or CDP, as detailed below.

STAB1 and CDP proteins have been characterized in the art. For example, various biological properties of STAB1 are disclosed in Dickinson et al., Cell 70: 631-645, 1992; Robertson et al., Genomics 23: 42-50, 1994; and Xu et al., Cytogenet. Cell Genet. 77: 205-206, 1997. Similarly, CDP examined and characterized in, e.g., Neufeld et al., Nat. Genet. 1: 50-55, 1992; Zeng et al., Gene 241: 75-85, 2000; Nepveu, Gene 270: 1-15, 2001; and Moon et al., Mol. Cell. Biol. 21: 6332-6345, 2001. Interaction between CDP and SATB1, and their role in tissue-specific regulation of gene expression, have been described in the art (see, e.g., Liu et al., Mol Cell Biol. 19: 4918-26, 1999). Polynucleotide and amino acid sequences encoding SATB1 or CDP from human and other animals, including their transcription variants, are all well known in the art. Exemplary STAB1 polynucleotide sequences include Accession Nos. NM_{—}002971 (human SATB1), BC001744 (human SATB1), AY413112 (human SATB1), AK127242 (human SATB1), NM_{—}009122 (mouse SATB1), AY413114 (mouse SATB1), BC011132 (mouse SATB1), XM_{—}236849 (rat SATB1). Examples of polynucleotide sequences encoding CDP proteins Accession Nos. NM_{—}181552 (human CDP), NM_{—}001913 (human CDP), NM_{—}181500 (human CDP), BC066592 (human CDP), NM_{—}198602 (mouse CDP), NM_{—}009986 (mouse CDP), and XM_{—}341053 (rat CDP). The corresponding amino acid sequences are also disclosed in the art.

In some embodiments, the SATB1 or CDP polypeptide used in the methods of the invention is a fragment that essentially consists of at least 15, often at least 20, 30, 40, or 50, 100 or more contiguous amino acids of an SATB1 or CDP protein encoded by any of the exemplary polynucleotide sequences provided above. Alternatively, the SATB1 or CDP polypeptides may have 60% identity, more often at least 70%, 80%, 85%, 90%, 95%, or greater identity to such an SATB1 or CDP protein. Typically, SATB1 or CDP fragments and variants for use in this invention retain the ability to repress transcription of genes that are under the control of an SATB1/CDP response element. Those SATB1 or CDP polypeptide variants or fragments that exhibit at least 50%, often 80%, 90%, 100% or greater activity relative to a reference SATB1 or CDP, e.g., human or mouse SATB1 or CDP, are typically used in the screening methods of the invention.

SATB1/CDP response elements are usually transcription regulatory elements that are recognized by SATB1 and/or CDP, e.g., an upstream transcription regulatory element of the IE gene of a herpesvirus that infect human cells. Various SATB1/CDP response elements can be employed to practice the screening methods of the present invention. In some embodiments, the SATB1/CDP response element used in the methods is the −593 to −549 region upstream of the transcription start site of human CMV IE gene (SEQ ID NO: 1). In some other embodiments, the −735 to −640 region (SEQ ID NO: 2) of the human CMV IE promoter, or a sub-region thereof, e.g., the −735 to −688 region (SEQ ID NO: 3) or the −687 to −640 region (SEQ ID NO: 4), can be used, as exemplified in the Examples below. Binding of an SATB1 polypeptide and/or a CDP polypeptide to an SATB1/CDP response element can be tested using assays well known to those of skill in the art. For example, a transcription assay that measures the ability of an SATB1 or a CDP polypeptide to repress a reporter gene expression under the control of an SATB1/CDP response element can be used.

Either naturally occurring or recombinant SATB1 or CDP polypeptides can be purified for use in the assays of the invention. Natural occurring SATB1 or CDP polypeptides can be purified from any source. Recombinant polypeptides can be purified from any suitable expression system. The polypeptides may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

An SATB1 or CDP polypeptide can then be used in a binding assay to identify test compounds that bind to the polypeptide. Binding of test compounds to an SATB1 or CDP polypeptide can be assayed by a number of methods including e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test compounds can be identified by detecting a direct binding to the SATB1 or CDP polypeptide, e.g., co-immunoprecipitation with the SATB1 or CDP polypeptide by an antibody directed to the SATB1 or CDP polypeptide. The test compound can also be identified by detecting a signal that indicates that the agent binds to the SATB1 or CDP polypeptide, e.g., fluorescence quenching.

Competition assays provide a suitable format for identifying test compounds that specifically bind to an SATB1 or CDP polypeptide. In such formats, test compounds are screened in competition with a compound already known to bind to the SATB1 or CDP polypeptide. The known binding compound can be a synthetic compound. It can also be an antibody, which specifically recognizes the SATB1 or CDP polypeptide, e.g., a monoclonal antibody directed against the SATB1 or CDP polypeptide. If the test compound inhibits binding of the compound known to bind the SATB1 or CDP polypeptide, then the test compound also binds the SATB1 or CDP polypeptide.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, “Antibodies, A Laboratory Manual,” Cold Spring Harbor Press (1988)); solid phase direct label RIA using ¹²⁵I label (see Morel et al., Mol. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified polypeptide bound to a solid surface or cells bearing either of these, an unlabelled test compound and a labeled reference compound. Competitive inhibition is measured by determining the amount of label bound to the solid surface or cells in the presence of the test compound. Usually the test compound is present in excess. Test compounds identified by competition assay include compounds binding to the same epitope as the reference compound and compounds binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize the SATB1 or CDP polypeptide or its fragments onto a solid phase matrix. The solid phase matrix is then put in contact with test compounds, for an interval sufficient to allow the test compounds to bind. After washing away any unbound material from the solid phase matrix, the presence of the compound bound to the solid phase allows identification of the compound. The methods can further include the step of eluting the bound compound from the solid phase matrix, thereby isolating the compound. Alternatively, other than immobilizing the SATB1 or CDP polypeptide, the test compounds are bound to the solid matrix and the SATB1 or CDP polypeptide molecule is then added.

Soluble assays include some of the combinatory libraries screening methods described above. Under the soluble assay formats, neither the test compounds nor the SATB1 or CDP polypeptide are bound to a solid support. Binding of an SATB1 or CDP polypeptide or fragment thereof to a test compound can be determined by, e.g., changes in fluorescence of either the SATB1 or CDP polypeptide or the test compounds, or both. Fluorescence may be intrinsic or conferred by labeling either component with a fluorophor.

In some embodiments, test compounds may be assayed for the ability to enhance or disrupt SATB1 or CDP interactions with proteins with which it forms complexes, e.g., with each other or with other homeodomain proteins such as PBX1. For example, a test compound can be added, either before, after, or concurrently, to binding reaction comprising SATB1 or CDP and PBX1. Modulation (e.g., stimulation) of the SATB1 or CDP binding interaction is achieved when the binding value deviates from that of the control by about 25%, optionally 50%, optionally 75%, 100% or more.

VI. Screening for Modulators of SATB1 or CDP Mediated Transcriptional Repression

To identify novel modulators of transcription and replication of herpesviruses (e.g., CMV), compounds that are shown to bind to SATB1 or CDP can be further tested for ability to enhance an activity relating to SATB1 or CDP-mediated transcriptional repression. Alternatively, test compounds can be examined directly for ability to modulate (e.g., enhance) such an activity. The compounds can be screened for ability to modulate (e.g., to stimulate) binding of SATB1 and/or CDP to an SATB1/CDP response element (e.g., a fragment of CMV IE promoter noted above). Such screening can be performed with any of the various DNA-protein binding assays described above, in the presence of the test compounds. More typically, the compounds are screened for ability to modulate transcription of a gene under the control of an SATB1/CDP response element (e.g., a reporter gene). In some preferred embodiments, a reporter expression construct harboring a reporter gene operably linked to an SATB1/CDP response element is employed to screen for modulators of SATB1 or CDP mediated transcriptional repression. The SATB1/CDP response element is typically from a herpesvirus that infects human cells, such as a transcription regulatory element of the CMV IE gene as described above. SATB1, CDP, as well as PDX1, are usually also present in the assay systems, either endogenously or recombinantly expressed (e.g., in THP-1 or HEK 293 cell nuclear extracts as detailed in the Examples below).

In in vivo assay systems, a host cell comprising the reporter construct is contacted with a test compound in the presence of SATB1 and/or CDP polypeptides. Preferably, both SATB1 and CDP are expressed in the host cell. In some of these embodiments, in addition to SATB1 and CDP1, PDX1 is also present in the host cell. These polypeptides can all be expressed endogenously in the host cells (e.g., HEK 293 cell or human monocytic cell line THP-1). Alternatively, it can be expressed from an expression vector that has been introduced into the host cell. The amount of time to effect potential interactions between the test compound and SATB1 and/or CDP may be empirically determined, such as by running a time course and measuring the level of transcription as a function of time.

To identify modulators that enhance SATB1 or CDP activities, host cells containing the reporter construct are treated with a test compound, and transcription activity of the reporter gene in the host cells is then measured. As a control, transcription of the reporter gene is also determined in host cells untreated with the test compound. The amount of transcription from the treated cells is then compared to the amount of transcription in the control cells. Any difference in the amount of transcription indicates that the test compound has in some manner altered the activity of the protein of interest. For example, a positive modulator of SATB1 or CDP activity is identified if the transcription level in the treated host cells is less than 75%, optionally 50%, 25%, 10%, 5%, 1% or less, of the transcription level in the control cells.

The amount of transcription can be measured by using any method known to those of skill in the art to be suitable. For example, transcription level of a reporter gene under the control of an SATB1/CDP response element can be examined using northern blots or PCR. In addition, expression level of the reporter molecule can be monitored by assaying the encoded polypeptide using, e.g., immunoassays. Transcription based assays using reporter genes can be used as described in the art, e.g., U.S. Pat. No. 5,436,128. The reporter genes can be, e.g., luciferase (as exemplified in the Examples below), chloramphenicol acetyltransferase, green fluorescent protein (GFP), β-galactosidase, and alkaline phosphatase.

Other than in vivo assay systems for identifying modulators of SATB1 or CDP-mediated transcription, in vitro assays may also be used. In such assays, a test compound is added to an in vitro transcription reaction that measures expression of a gene that is regulated by SATB1 or CDP (e.g., the CMV IE gene) or a reporter gene under the control of an SATB1/CDP response element. Typically, an SATB1 and/or a CDP polypeptide is also present in the reaction in order to assess the ability of a test compound to modulate (e.g., enhance) SATB1 or CDP-mediated transcriptional repression.

Expression vectors that express an SATB1 or CDP polypeptide and reporter gene under the control of an SATB1/CDP response element can be generated using procedures well known in the art (see, e.g., Sambrook et al., and Ausubel et al., both supra). The cellular transcription assays can be performed using any host cell that can support transcription of SATB1 or CDP and the reporter gen. Examples of suitable cell lines include HEK 293T cells as discussed in the Examples below. Additional cell lines that may be employed include HEK 293, HCT116 cells, MCF-7 cells, Hela cells, THP-1, and HepG2 cells.

VII. Therapeutic Applications

The present invention provides compositions and methods for treating infections of herpesviruses in various subjects including human. There are a number of diseases and conditions that are mediated by or associated with herpesviruses. For example, human CMV is associated with infectious mononucleosis. Infection by human herpesvirus 6 & 7 leads to mild early childhood roseola. Herpes simplex virus 1 causes facial, labial and ocular lesions, while herpes simplex virus 2 is associated with genital lesions. Varicella-zoster virus (human herpesvirus 3) is implicated in chickenpox and shingles. Epstein-Barr virus (human herpesvirus 4) is a cofactor in human cancers, and Karposi's sarcoma herpesvirus (human herpesvirus 8) is a cofactor in Karposi's sarcoma.

Human CMV is also a common cause of mental retardation in children who acquire the infection in utero from mothers carrying an active infection. In addition, some newborn infants carry CMV and the virus can cause severe congenital disease in the fetus or infant. Inhibiting or modulating the IE expression and/or function has been suggested in the art as novel strategy of treating herpesvirus (e.g., CMV) associated diseases (see, e.g., Scholz et al., Antiviral Res. 49: 129-145, 2001). All the diseases and conditions discussed above can be treated with the novel modulators of the present invention which enhance SATB1 and/or CDP-mediated transcriptional repression of the IE gene.

Modulators that enhance or stimulate SATB1/CDP mediated transcription repression activity can be administered directly to a subject (e.g., a human) that is infected by a herpesvirus (e.g., CMV). The modulators can be administered alone or as the active ingredient of a pharmaceutical composition. Administration can be by any of the routes which are well known to those of skill in the art and which are normally used for introducing a modulating compound into ultimate contact with the tissue to be treated.

The identified stimulator of SATB1/CDP-mediated transcriptional repression can be administered to a patient at therapeutically effective doses to prevent, treat, or control herpesviral disease, e.g., CMV infection. The compounds are administered to a patient in an amount sufficient to elicit an effective protective or therapeutic response in the patient. An effective protective or therapeutic response is a response that at least partially arrests or slows the symptoms or complications of the disease. An amount adequate to accomplish this is defined as “therapeutically effective dose.” The optimal dose level for any patient will depend on a variety of factors including the efficacy of the specific modulator employed, the age, body weight, physical activity, and diet of the patient, and on a possible combination with other drug. The size of the dose also will be determined by the existence, nature, and extent of any adverse side-effects that accompany the administration of a particular compound or vector in a particular subject.

In determining the effective amount of the modulator to be administered, a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the modulator at various concentrations, as applied to the mass and overall health of the subject. Administration can be accomplished via single or divided doses.

The modulators of the invention may be used alone or in conjunction with other agents that are known to be beneficial in treating or preventing human diseases that are mediated by herpesviruses, e.g., CMV-1 infection. The modulators of the invention and another agent may be co-administered, either in concomitant therapy or in a fixed combination, or they may be administered at separate times.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington: The Science and Practice of Pharmacy, Mack Publishing Co., 20^{th} ed., 2000).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, intrathecally or into the eye (e.g., by eye drop or injection). The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

Pharmaceutically acceptable carriers are determined in part by the particular composition being administered, as well as by the particular method used to administer the composition. Accordingly, there are a wide variety of suitable formulations of pharmaceutical compositions of the present invention, as described below (see, e.g., Remington: The Science and Practice of Pharmacy, Mack Publishing Co., 20^{th} ed., 2000).

### EXAMPLES

The following examples are offered to illustrate, but not to limit the scope of the present invention.

### Example 1

### General Methods and Materials

Cells and reagents. The human monocytic cell line, THP-1, was obtained from American Type Culture Collection. Differentiation of THP-1 cells was achieved with supplementation of growth media with 10 nM phorbol 12-myristate 13-acetate (PMA; Sigma) for 2 days. The CMV-Luc stable cell line, 293-CMV-Luc, was generated as described in (Chao et al., J. Biol. Chem., 279: 16111-120, 2004).

Plasmids and plasmid construction. The CMV-CDP plasmid (i.e. pRc/CMV CDP) was described in Liu et al., Mol. Cell Biol. 19: 4918-4926, 1999. This plasmid was subcloned into pUB6 (Invitrogen) to generate pUB-CDP, in which the expression of CDP was driven by the human ubiquitin C promoter. The pCITE-CDP plasmid was created by cloning the coding regions of CDP into the pCITE vector (Novagen) using CMV-CDP as the templates. The SATB1 cDNA was obtained from internal clone collections and used to create pUB-SATB1 and pCITE-SATB1. The pUB-PDX1, pCITE-PDX1, and pUB-F-Luc (ubiquitin-firefly luciferase) plasmids were constructed as described in (Chao et al., J. Biol. Chem., 279: 16111-120, 2004). CMV-Renilla luciferase (CMV₋₇₃₅-Luc or pRL-CMV) was purchased from Promega, which contains the promoter region of CMV IE gene from nucleotide −735 to +61. CMV₋₅₄₉-Luc, which the unique region of the CMV IE promoter was deleted, was also utilized in the study. The DNA fragment of CMV IE promoters from position −549 to +61 was generated by PCR and then replaced the nucleotides from −735 to +61 of CMV₋₇₃₅-Luc to generate the CMV₋₅₄₉-Luc plasmid. The pCITE constructs were used in in vitro reactions (TNT Quick Coupled Transcription/Translation Systems, Promega) to generate the in vitro synthesized proteins.

EMSAs. The different DNA fragments of the unique region of CMV IE promoters were used as the DNA probes for EMSAs. CMV_{-593 to -549} contains the 45-bp PDX1 binding elements (5′-GGCATTGATTATTGACTAGTTATTAATAGTAATCAATTACGGGGTCATTAGTTCA) (SEQ ID NO: 5), whereas CMV_{-735 to -688} (5′-TTGAATCAATATTGGCCATTAGCCATATTATTCATTGGTTATATAGCATAAATCA ATA) (SEQ ID NO: 6), CMV_{-687 to -640} (5′-TAAATCAATATTGGCTATTGGCCATTGCATACGTTGTATCTATATCATAATATGTA CA) (SEQ ID NO: 7), and CMV_{-639 to -594} (5′-TATATGTACATTTATATTGGCTCATGTCCAATATGACCGCCATGTTGGCATTGATT ) (SEQ ID NO: 8) contain the rest parts of the unique regions. EMSAs were performed as described in (Chao et al., J. Biol. Chem., 279: 16111-120, 2004). The anti-PDX1, anti-SATB1, anti-CDP, anti-Brn-3.0, and anti-SP1 antibodies were purchased from Santa Cruz Biotechnology, Inc.

Transfection and luciferase assays. 293 or 293T cells were grown to 50-80% confluence in 96-well plates. Transfections were performed using Fugene 6 (Roche) as described in the manufacturer's manuals. Cells were co-transfected with a CMV-R-Luc plasmid (CMV₋₇₃₅s-Luc or CMV₋₅₄₉-Luc), the indicated expression vectors (i.e. pUB-PDX1, -SATB1, or -CDP) and a pUB-F-Luc internal control plasmid. Firefly and Renilla luciferases were measured at 48 hours post transfection using the Dual-Glo assay system (Promega) and the activities determined using an Acquest multimode reader (LJL Biosystems, Inc.).

### Example 2

### SATB1 and CDP Associate with PDX1 Binding Site of CMV IE Promoter

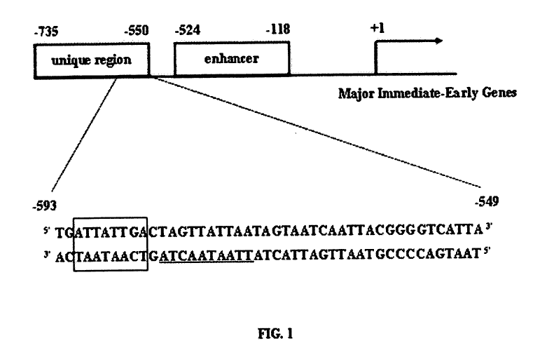

PDX1 binds to a 45-nucleotide fragment located at position −593 to −549 upstream of the transcription start site of the human CMV IE gene (FIG. 1) and represses the activity of the CMV IE promoter in 293/293T cells (Chao et al., J. Biol. Chem., 279: 16111-120, 2004). A putative Brn-3.0 binding site, T(A/T)AATNANT(A/T) (SEQ ID NO: 9), was found to be located between the inverted sequences −582 and −573 of CMV IE promoter (Turner et al., Nucleic Acids Res. 25: 2589-2594, 1997).

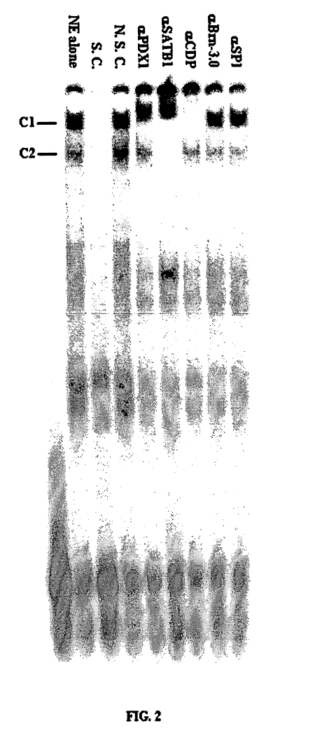

To determine the possible involvement of CDP, SATB1, and Brn-3.0 proteins in CMV IE gene regulation, EMSAs were carried out using HEK 293 nuclear extracts and the CMV_{-593 to -549} DNA probe, which contains the entire 45-bp PDX1 binding region. As shown in FIG. 2, the formation of two specific DNA-protein complexes, C1 and C2, was observed. Incubation of nuclear extracts with antibodies raised against PDX1 and CDP proteins resulted in disruption of the C1 complex and has no significant effects on formation of the C2 complex (FIG. 2). Disruption of both C1 and C2 complexes were observed when the anti-SATB1 antibodies were included in the reaction, whereas incubation of the anti-Brn-3.0 antibodies did not have any detectable effects (FIG. 2). The data of EMSAs not only suggested that SATB1 and CDP are the additional cellular proteins which bind to the 45-bp region of the human CMV IE promoter, but also indicated that PDX1/SATB1/CDP are the cellular proteins present in the C1 complex and SATB1 is the protein component of the C2 DNA-protein complex.

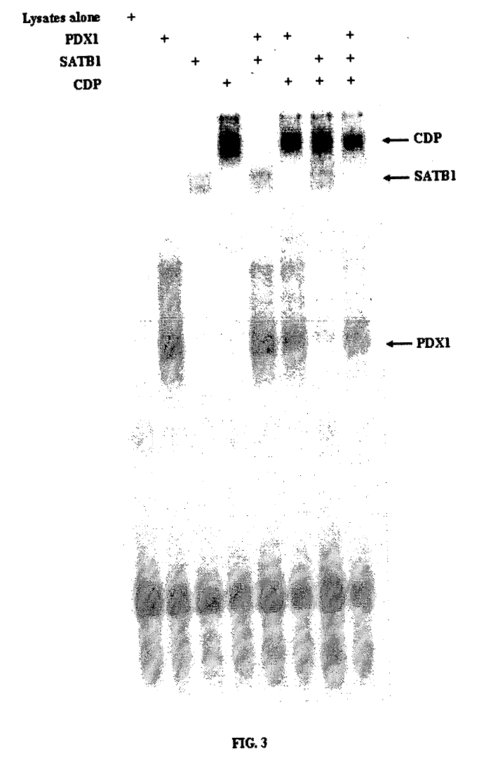

To confirm the associations between the 45-bp CMV element and SATB1/CDP proteins, EMSAs were carried out using in vitro synthesized SATB1 and CDP and the CMV_{-593 to -549} probe (FIG. 3). Just like the positive control, in vitro translated PDX1 proteins, SATB1 and CDP associated with the 45-bp region (FIG. 3). Two DNA-protein complexes were observed in the reactions containing in vitro synthesized SATB1 (FIG. 3). It has been reported that SATB1 can form a homodimer itself. Thus, it is likely that SABT1 can bind to the 45-bp DNA as a monomer and homodimer (FIG. 3).

We next examined the possible interaction between these three proteins in association with the DNA. EMSAs were performed with the indicated combinations of PDX1, SATB1, and CDP proteins included in the reactions (FIG. 3). The results showed that PDX1, CDP, and SATB1 did not bind to the CMV DNA as a heterodimer or heterotrimer but still associated with the DNA individually in vitro (FIG. 3).

### Example 3

### PDX1, SATB1, and CDP Bind to Human CMV IE Promoter

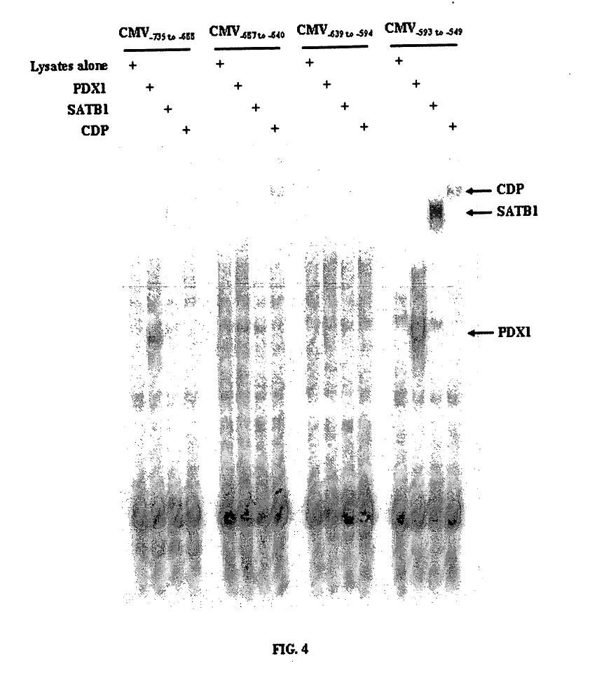

The 45-bp region of interest contains a high AT content (73%; FIG. 1). Indeed, the whole unique region (nucleotides from −735 to −550) is AT-rich with a 68% AT content. Since PDX1, SATB1, and CDP proteins intend to bind to the AT-rich regions, the association between the unique region and the three proteins was further investigated. The unique region was equally divided into 4 fragments, CMV_{-735 to -688}, CMV_{-687 to -640}, CMV_{-639 to -594}, and CMV_{-593 to -549}, and used as the DNA probes in EMSAs incubating with the indicated in vitro synthesized protein. In addition to the CMV_{-593 to -549} region, all three proteins bound to CMV_{-735 to -688} with lower affinity (FIG. 4). CDP also associated with the CMV_{-687 to -640} element whereas none of the proteins bound to CMV_{-639 to -594} (FIG. 4). We also performed the same set of experiments as shown in FIG. 3 to determine if the three proteins were able to bind to the DNAs cooperatively (i.e. CMV_{-735 to -688}, CMV_{-687 to -640}, and CMV_{-639 to -594}) as a heterodimer or heterotrimer. The results showed that the in vitro synthesized PDX1, SATB1, and CDP only bind to the DNAs (i.e. CMV_{-735 to -688} and CMV_{-687 to -640}, but not CMV_{-639 to -594}) as a monomer, instead of a heterodimer or heterotrimer.

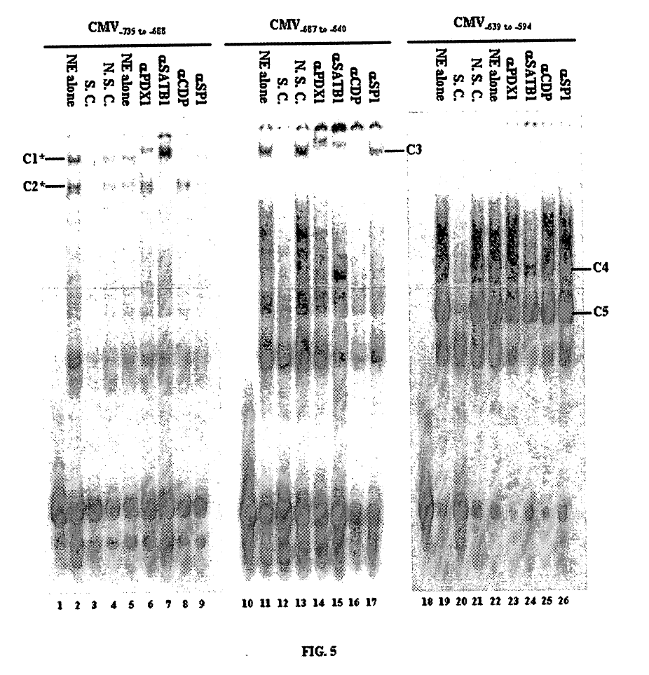

To confirm the binding between PDX1/SATB1/CDP and the unique region of the CMV IE promoter, EMSAs were carried out using 293 nuclear extracts and the antibodies against PDX1, SATB1, and CDP. Two specific DNA-protein complexes were observed when CMV_{-735 to -688} was used as the DNA probe (FIG. 5; “C1*” and “C2*”). The anti-PDX1 and anti-CDP antibodies resulted in a supershift and disruption of the complex C1* respectively, whereas the formation of the complex C2* was completely disrupted when the anti-SABT1 antibody was included in the reaction (FIG. 5). In addition, the anti-SATB1 antibody also caused the supershifted complex C1* (FIG. 5). In agreement with the EMSAs using in vitro translated proteins (FIG. 5), these data demonstrated the association between PDX1/SATB1/CDP and CMV_{-735 to -688} and indicated that PDX1/SATB1/CDP were the major components of the C1* complex and SATB1 was present in the C2* complex. Furthermore, the sizes and protein components of C1* and C2* complexes are very similar to those of C1 and C2 complexes in which CMV_{-593 to -549} was used as the DNA probe (FIG. 2 and FIG. 5).

The same EMSAs were performed using CMV_{-687 to -640} as the DNA probe. A specific DNA-protein complex, C3, was detected while incubating with 293 nuclear extracts. The C3 complex was significantly disrupted or supershifted with the incubation of anti-PDX1, anti-SATB1 and anti-CDP antibodies, suggesting that PDX1, SATB1, and CDP were the protein components of the specific DNA-protein complex (FIG. 5). However, results from the EMSAs using in vitro synthesized proteins demonstrated that only CDP, but not SATB1 or PDX1, was able to bind to the CMV_{-687 to -640} region (FIG. 4). It is possible that the post-translational modifications, which are missing in the in vitro translated PDX1 and SATB1 proteins, might be required for PDX1 and SATB1 binding to the CMV_{-687 to -640} element in vivo.

The identical set of EMSAs were carried out using CMV_{-639 to -594} DNA probe. Two specific complexes, C4 and C5, were detected (FIG. 5). Neither of the DNA-protein complexes contained PDX1, SATB1, or CDP proteins (FIG. 5), which was consistent with the results obtained from EMSAs using in vitro translated proteins (FIG. 4). Collectively, the EMSAs using nuclear extracts confirm that there are additional PDX1, SATB1, and CDP binding sites present within the unique regions of human CMV IE promoters.

### Example 4

### PDX1/SATB1/CDP Repress CMV IE Promoter Additively

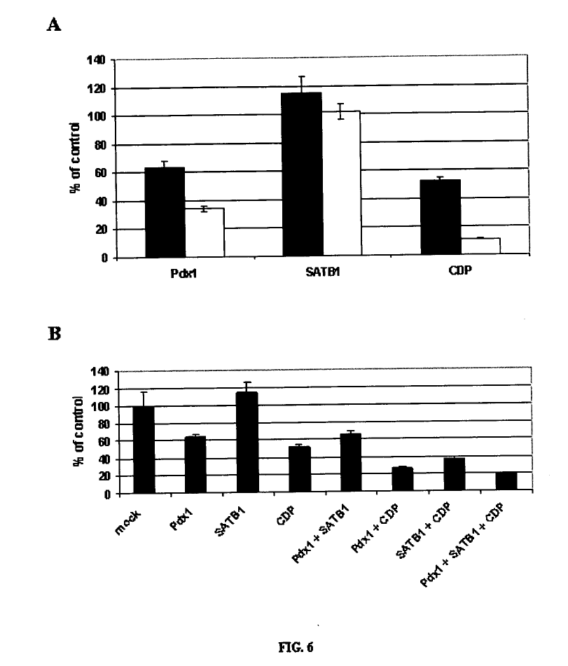

We next examined the biological functions of PDX1/SATB1/CDP on the regulation of the CMV IE promoter. HEK 293T cells were transiently co-transfected with the CMV-Luc reporter and an expression plasmid encoding PDX1, SATB1, or CDP (FIG. 6A). As with PDX1, overexpression of CDP inhibited the activity of the CMV-IE promoter (FIG. 6A). Furthermore, a dose-dependent inhibition of luciferase activity was observed when PDX1 and CDP were overexpressed (FIG. 6A). However, overexpression of SATB1 did not cause any significant effects on the reporter gene (FIG. 6A). We further examined the functional interaction between PDX1, SATB1, and CDP, and the effects on CMV IE-dependent transcription. Additive effects were observed when cells were co-transfected with PDX1/CDP, SATB1/CDP, and PDX1/SATB1/CDP expression constructs, resulting in greater inhibition on CMV IE-dependent transcription (FIG. 6B). Similar results were obtained when 293 and CMV-Luc stable cells (i.e. 293-CMV-Luc) were used.

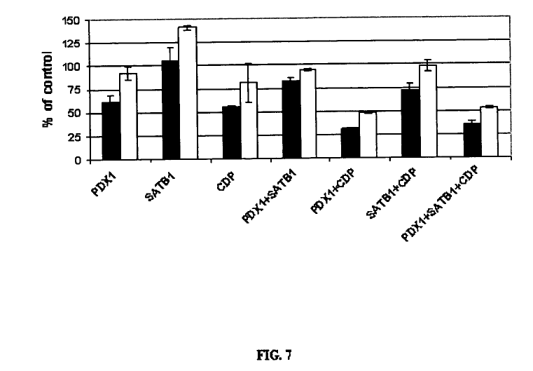

The mutant CMV-Luc construct (i.e. CMV₋₅₄₉-Luc), in which the entire unique region was deleted, was generated and transiently transfected in 293, 293T and HeLa cells. The deletion resulted in up to 2-fold induction in luciferase expression. We further investigated the effects of overexpressing PDX1/SATB1/CDP on CMV₋₅₄₉-Luc in 293 cells. As shown in FIG. 7, the overexpression of PDX1/SATB1/CDP had more modest effects on the activity of the CMV IE promoter when the entire unique region was deleted. Collectively, our data demonstrate that PDX1, CDP, and SATB1, bind to the unique region of human CMV IE promoter and collaboratively repress the transcription of CMV IE-dependent transcription in 293 and 293T cells.

### Example 5

### PDX1/SATB1/CDP Bind to CMV IE Promoter in Monocytes

293 and 293T cells are non-permissive cell lines for human CMV, but allow the transcription of CMV IE genes. To investigate the involvement of PDX1, SATB1, and CDP in transcriptional regulation of CMV, the interaction between the unique region and the three homeoproteins were examined in a human monocytic cell line, THP-1. CMV IE genes do not express in the undifferentiated THP-1 cells. However, once the differentiation of THP-1 cells are induced, the transcriptional blocks are removed resulting in reactivation of CMV IE genes. Nuclear extracts prepared from the undifferentiated and differentiated THP-1 cells were analyzed in EMSAs using CMV_{-593 to -549} as the DNA probe. Two specific DNA-protein complexes were formed in undifferentiated THP-1 cells (FIG. 8; S1 and S2). The supershifted complexes were detected when the anti-SATB1 antibodies were included in the reaction (FIG. 8; TH-1). No significant effects on the complexes were observed with incubation of anti-PDX1 and anti-CDP antibodies (FIG. 8; THP-1). As noted, the sizes of complexes S1 and S2 were similar to those of DNA-in vitro synthesized SATB1 complexes shown in FIG. 3, suggesting that SATB1 was the only protein present in the complexes and might bind to the DNA as a monomer and homodimer. While the nuclear extracts of the PMA-treated THP-1 cells were utilized in EMSAs, the formation of a novel DNA-protein complex was observed (FIG. 8; arrow). Interestingly, the size of the new complex was similar to that of complex C1 formed in 293 cells, and both complexes contained PDX1, SATB1, and CDP proteins (FIGS. 2 and 8). In addition, the differentiation of THP-1 cells disrupted the formation of complex S2 significantly (FIG. 8).

A series of EMSAs were performed using THP-1 nuclear extracts and the different DNA fragments of the unique region as the DNA probes. Compared with EMSAs shown in FIG. 8, similar results were obtained when CMV_{-735 to -688} was used as the probe (FIG. 9A). Two SATB1 containing complexes, S1* and S2*, were detected in the undifferentiated THP-1, and a newly formed complexes which contained PDX1/SATB1/CDP were observed using the nuclear extracts prepared from the differentiated THP-1 (FIG. 9A). Furthermore, the S1* complex was the predominant DNA-protein complex once THP-1 differentiation was induced (FIG. 9A).

Two specific DNA-protein complexes, M1 and M2, were detected in the EMSAs using THP-1 nuclear extracts and the CMV_{-687 to -640} DNA probe. M1 complex was predominant in THP-1 cells whereas M2 complex was the major specific form in the PMA-treated cells (FIG. 9B). PDX1, SATB1, and CDP were the protein components of both M1 and M2 complexes (FIG. 9B). An identical set of gel shift assays were carried out using the CMV₋₆₃₉₋₅₉₄ DNA as the probe. Two specific DNA-protein complexes, which did not contain PDX1, SATB1, or CDP proteins, were observed. No formation of novel DNA-protein complexes were detected when THP-1 was induced to differentiate. In addition, the sizes of these two complexes were similar to those of the C4 and C5 complexes observed in the EMSAs using 293 nuclear extracts (FIG. 5). The data of EMSAs using the nuclear extracts prepared from undifferentiated and differentiated monocytes demonstrate the possible involvement of PDX1, SATB1, and CDP in regulating the activity of human CMV IE promoter in human CMV host cells. The results further indicate that SATB1 is a major cellular protein associating with the unique region of the CMV IE promoter in the IE gene-inactive THP-1 cells.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, sequence accession numbers, patents and patent applications cited herein are hereby expressly incorporated by reference in their entirety and for all purposes as if each is individually so denoted.

We claim:

1. A method for identifying a compound that inhibits replication of a herpesvirus that infects human cells, the method comprising screening test compounds for ability to enhance SATB1 or CDP-mediated transcriptional repression of the herpesvirus, thereby identifying a compound that inhibits replication of the herpesvirus.

2. The method of claim 1, wherein the herpesvirus is human cytomegalovirus (CMV).

3. The method of claim 1, wherein the screening comprises (a) contacting test compounds with an SATB1 or a CDP polypeptide, and a polynucleotide comprising an SATB1/CDP response element; and (b) identifying a compound that enhances binding to the SATB1/CDP response element by the SATB1 or the CDP polypeptide relative to the binding in the absence of the compound.

4. The method of claim 3, further comprising testing the compound thus identified for ability to inhibit replication of the herpesvirus.

5. The method of claim 1, wherein the screening comprises (a) contacting test compounds with an SATB1 or a CDP polypeptide, and a gene under the control of an SATB1/CDP response element, and (b) identifying a compound that reduces expression level of the gene relative to expression level of the gene in the absence of the compound.

6. The method of claim 5, wherein the test compounds are pre-screened for ability to specifically bind to the SATB1 or the CDP polypeptide.

7. The method of claim 5, wherein expression level of the gene is measured using a reporter construct comprising the SATB1/CDP response element operably linked to a polynucleotide that encodes a detectable label.

8. The method of claim 5, wherein the SATB1/CDP response element comprises a transcription regulatory sequence from the IE gene of the herpesvirus.

9. The method of claim 8, wherein the herpesvirus is human cytomegalovirus (CMV), and the SATB1 or CDP polypeptide is human SATB1 or CDP.

10. The method of claim 8, wherein the transcription regulatory sequence comprises nucleotides −593 to −549 (SEQ ID NO: 1), nucleotides −735 to −688 (SEQ ID NO: 3), or nucleotides −687 to −640 (SEQ ID NO: 4) of human CMV IE gene promoter.

11. A method for identifying a compound that inhibits replication of a herpesvirus that infects human cells, the method comprising (a) contacting test compounds with an SATB1 or a CDP polypeptide, and a reporter gene operably linked to an SATB1/CDP response element, and (b) identifying a compound that reduces expression level of the reporter gene in the presence of the compound relative to expression level of the reporter gene in the absence of the compound, thereby identifying a compound that inhibits replication of the herpesvirus.

12. The method of claim 11, wherein the SATB1/CDP response element comprises a transcription regulatory sequence from the IE gene of the herpesvirus.

13. The method of claim 11, wherein the SATB1/CDP response element comprises nucleotides −593 to −549 (SEQ ID NO: 1), nucleotides −735 to −688 (SEQ ID NO: 3), or nucleotides −687 to −640 (SEQ ID NO: 4) of human CMV IE gene promoter.

14. The method of claim 11, wherein the reporter gene and the SATB1/CDP response element are present in an expression vector.

15. The method of claim 11, wherein the contacting is in a host cell expressing the SATB1 and the CDP polypeptide.

16. The method of claim 15, wherein the SATB1 and the CDP polypeptide are expressed from a second expression construct that has been introduced into the host cell.

17. The method of claim 15, wherein the host cell expresses the SATB1 and the CDP polypeptides endogenously.

18. The method of claim 15, wherein the reporter gene is a luciferase gene.

19. The method of claim 15, wherein the host cell is HEK 293 cell.

20. A method of inhibiting replication of a herpesvirus in a human subject, the method comprising administering to the subject a pharmaceutical composition comprising an effective amount of a compound that inhibits replication of the herpesvirus, wherein the compound is identified by screening test compounds for ability to enhance SATB1 or CDP-mediated transcriptional repression of the herpesvirus.

21. The method of claim 20, wherein the herpesvirus is human CMV.

22. The method of claim 20, wherein the compound enhances SATB1 or CDP-mediated transcriptional repression of the IE gene of the herpesvirus.


SEQUENCE LISTING (9 sequences)

SEQ ID NO: 1 - DNA, 45 bp, Artificial Sequence
  Synthetic Sequence
tgattattgactagttattaatagtaatcaattacggggtcatta

SEQ ID NO: 2 - DNA, 96 bp, Artificial Sequence
  synthetic sequence
tcaatattggccattagccatattattcattggttatatagcataaatcaatattggctattggccattgcatacgttgtatctatatcataatat

SEQ ID NO: 3 - DNA, 48 bp, Artificial Sequence
  synthetic sequence
tcaatattggccattagccatattattcattggttatatagcataaat

SEQ ID NO: 4 - DNA, 48 bp, Artificial Sequence
  synthetic sequence
caatattggctattggccattgcatacgttgtatctatatcataatat

SEQ ID NO: 5 - DNA, 55 bp, Artificial Sequence
  synthetic sequence
ggcattgattattgactagttattaatagtaatcaattacggggtcattagttca

SEQ ID NO: 6 - DNA, 58 bp, Artificial Sequence
  synthetic sequence
ttgaatcaatattggccattagccatattattcattggttatatagcataaatcaata

SEQ ID NO: 7 - DNA, 58 bp, Artificial Sequence
  synthetic sequence
taaatcaatattggctattggccattgcatacgttgtatctatatcataatatgtaca

SEQ ID NO: 8 - DNA, 56 bp, Artificial Sequence
  synthetic sequence
tatatgtacatttatattggctcatgtccaatatgaccgccatgttggcattgatt

SEQ ID NO: 9 - DNA, 10 bp, Artificial Sequence
  synthetic sequence
twaatnantw